(19) United States
(12) Patent Application Publication
(10) Pub. No.: US 2021/0263016 A1
(43) Pub. Date: Aug. 26, 2021

(54) EVALUATING THE EFFICACY OF LEAVE-ON COSMETIC COMPOSITIONS TO PROTECT FROM POLLUTANTS

(71) Applicant: CONOPCO, INC., D/B/A UNILEVER, Englewood Cliffs, NJ (US)

(72) Inventors: Chengdong JI, Shanghai (CN); Sheng MENG, Shanghai (CN); Shangchun Yl, Shanghai (CN); Caigen YUAN, Shanghai (CN)

(73) Assignee: Conopco, Inc., d/b/a UNILEVER, Trumbull, CT (US)

(21) Appl. No.: 17/053,825

(22) PCT Filed: May 2, 2019
(86) PCT No.: PCT/EP2019/061192

§ 371 Date: Nov. 9, 2020

(30) Foreign Application Priority Data
May 17, 2018 (CN) PCT/CN2018/087260
Jul. 2, 2018 (EP) 18181178.7

(51) Int. Cl. G01N 33/50; G01N 21/65; G01N 21/64

(52) U.S. Cl. CPC G01N 33/5082; G01N 2021/656; G01N 21/6458; G01N 21/65

(57) ABSTRACT

Disclosed is an in-vitro method to determine efficacy of a cosmetic composition or of one or more ingredients comprised therein to inhibit a particulate pollutant from contacting skin, said method comprising the steps of: (i) contacting a human skin equivalent with a cosmetic composition to form, upon drying, a layer thereof extending along mutually perpendicular X and Y and Z axis where along said Z axis said layer is 1 to 100 μm, where said Z axis indicates thickness of said layer; (ii) depositing, on said layer, a known amount of a model fine particulate matter comprising a first substance responsive to depth profile analysis where said cosmetic composition comprises a second substance, not comprised in said model fine particulate matter but also responsive to said depth profile analysis where the response of said first and second substances are distinguishable; (iii) for a pre-determined period after depositing said model fine particulate matter, periodically measuring response of said first and said second substance by said depth profile analysis, to thereby determine the amount of said model fine particulate matter at defined intervals along said Z axis; and, (iv) ascertaining said efficacy based on the amount of said model fine particulate matter at said defined intervals along said Z axis throughout said pre-determined period.

### FIELD OF THE INVENTION

The present invention relates to a method of evaluating efficacy of cosmetic compositions. More particularly, the invention relates to a method for evaluating efficacy of leave-on cosmetic compositions to prevent or inhibit particulate pollutants from contacting skin.

### BACKGROUND OF THE INVENTION

The World Health Organization (WHO) reports that outdoor air pollution originates from natural and anthropogenic sources. While natural sources contribute substantially to local air pollution in arid regions more prone to forest fires and dust storms, the contribution from human activities far exceeds natural sources.

Such human activities include fuel combustion, heat and power generation and industrial facilities (e.g. manufacturing factories, mines, and oil refineries). WHO classifies pollutants into particulate matter, black carbon, ground-level ozone and oxides of carbon, nitrogen and sulphur.

Particulate matter (PM) are inhalable particles composed of sulphate, nitrates, ammonia, sodium chloride, black carbon, mineral dust and water. Particles with a diameter of less than 10 microns (PM₁₀), including fine particles less than 2.5 microns (PM_{2.5}) pose the greatest risks to health, as they can enter the lungs and the bloodstream. Carbon black (soot) and dust (mineral oxides, such as iron oxides and the like) comprise much of the particulate matter in these size ranges.

WHO defines air pollution as contamination of indoor or outdoor environments by any chemical, physical, or biological agent that modifies the natural characteristics of the atmosphere. The U.S. Environmental Protection Agency (EPA) and the WHO have summarized the global extent of common atmospheric pollutants. In addition, there are innumerable reports and scientific publications pertaining to adverse effects of pollution on human skin. These adverse effects include premature ageing, development of fine lines and wrinkles, pigmented spots, hyperpigmentation, rash and inflammation.

Some cosmetic compositions claim to prevent, inhibit or restrict the particulate pollutants from contacting human skin by forming a protective layer, i.e., the ability to partly or fully block environmental pollutants like particles, oxide/superoxides and gases from contacting human skin. Formulation scientists often find it necessary to be able to substantiate such claims with evidence. Therefore, several manufacturers and researchers have published their own methods of testing/analysing or verifying the efficacy of such compositions. A purpose of such methods is to ascertain the efficacy of a candidate cosmetic composition. At times the purpose also is to compare the efficacy of one or more compositions or active ingredients, e.g. polymers, and rank them accordingly. Some of these tests are conducted on human volunteers. Some others have been conducted on suitable skin-equivalents such as plastic membranes, living-skin equivalents, Vitro-Skin®, in vitro skin models, ex vivo skin and the like. While human skin-equivalents is one components of such test methods, selection of an appropriate pollutant is equally important. However, it is not always possible to perform tests with real-life pollutants therefore model pollutants are often used.

DE4340827 C1 (Aerochemica, 1995) discloses a method and device for in-vitro determination of efficacy against chemical substances and substance mixtures. The barrier effect of a cosmetic is determined in a well-defined amount and thickness on a predetermined surface of a skin simulating membrane, like cellulose nitrate, nylon or PTFE. The concerned composition is applied to this membrane. Subsequently, the membrane is sandwiched on a specific dry indicator paper laid and stretched while maintaining flat contact with the membrane. Then, a measured drop of the defined pollutant is applied over the layer of the cosmetic. Depending on the effectiveness of the preparation, the pollutant may permeate rapidly and trigger a colour change in the indicator paper. This change is monitored by irradiating white light via fibres onto the indicator paper by means of a statistically split optical fibre bundle. The remaining, about 50% receiver fibres absorb the scattered light and lead it to a detector. Here there is signal filtering and amplification followed by recording. For example, a photodiode receives the light from the fibre optic cable, an amplifier amplified linearly or logarithmically (depending on the colour change of the indicator paper) and gives the signal through the output to a recorder.

The method disclosed in DE4340827 C1 relies on the use of visual colour-changing indicators therefore using this method it is possible to find out when the colour changes which tells the point in time at which the membrane is no longer able to provide barrier effect.

Zhai et al have disclosed an in vivo method in Contact Dermatitis, 1996, 35, 92-96, to measure the effectiveness of skin protective creams against two dye indicator solutions: methylene blue in water and oil red in ethanol, representative of model hydrophilic and lipophilic compounds. Three commercial compositions were assayed by measuring the dye in cyanoacrylate strips of protected skin samples after various application times. The flexural surfaces of the forearms of 6 normal volunteers (3 female and 3 male, mean age 26.8 years) were treated.

Solutions of 5% methylene blue in water and 5% oil red in ethanol are prepared, and applied to untreated skin and protective-cream pre-treated skin with the aid of aluminium occlusive chambers, for 0 hours and 4 hours, respectively. At the end of the application time, the creams are removed. Consecutive skin surface biopsies (SSB) from I to 4 strips were taken. The amount of stain in each strip was determined by colorimetry (Chroma Meter CR 300), and the cumulative amount of stain from 1 to 4 strips in each measurement was calculated. The cumulative amount represents the amount of permeation of each solution at each time point, and the efficacy of skin barrier cream.

A somewhat similar method of Marks et.al, is disclosed in British Journal of Dermatology (1989) 120, 655-6.

Nizard et.al have disclosed a method in H&PC Today, 10 (1) January/February 2015. In this method, a skin explant is exposed under a patch to 32 pollutants (27 heavy metals and 5 hydrocarbons) as an ex-vivo model for pollution damage. The author's formula is claimed to protect against pollution damage (skin morphology integrity scoring) and lipids peroxidation (by Malondialdehyde measurement). As their formula is applied on explants before the patch with pollutants, the formula creates a physical barrier.

Dow Corning has disclosed a test method to quantify the extent of protection conferred by its product, Splash Shield®, against particulate adhesion. A thin film of the test material is formed on collagen followed by surface analysis and exposure to carbon black repeat analysis.

Further, Dow Corning has disclosed yet another test method in which a test material is coated on a synthetic substrate through which ozone can diffuse. At the other end is a receptacle containing solution of a dye which changes colour upon contact with ozone. Intensity of the colour is inversely proportional to the protection offered by the test material.

BASF has disclosed an ex-vivo model to determine the efficacy of its product named Purisoft® against cigarette smoke. The method relies on organotypic cultures of human skin on which the concerned products are applied followed by exposure to smoke. This is followed by biopsies and confocal microscopy to determine the extent of protection offered by the compound of interest.

Lipotec has disclosed a somewhat similar method for a similar purpose about their product Pollushield®.

In Contact Dermatitis, 1996, 35, 219-225, Olivarius et.al. have disclosed a method which relies on the colour of Crystal violet which binds firmly to keratin (stratum corneum) when painted on the skin. When the skin is pretreated with a water-repellant cream, the penetration of aqueous solution of crystal violet is impaired, leading to lesser binding and paler colour. The relative efficacy of different creams is evaluated visually by comparing intensities quantified by measurement of skin reflectance. Low reflectance indicates a high binding. The protection offered by the cream is calculated as the additional colour resistance induced by the cream (x-y), divided by the maximal additional colour resistance obtainable (100−y).

EP1760440 A1 (P&G) discloses a method comprising the steps of: selecting a Raman-active substance linked to the effectiveness of the skin care composition to be determined;
(i) measuring the concentration profile of said Raman-active substance as a function of depth within a test area of skin using Confocal Raman spectroscopy;
(ii) determining the thickness of the stratum corneum within said test area; then
(iii) applying the skin care composition to said test area; then
(iv) measuring the concentration profile of said Raman-active substance as a function of depth within said test area using Confocal Raman spectroscopy;
(v) determining the thickness of the stratum corneum within said test area; then
(vi) calculating the effectiveness of the skin care composition as a function of both; the concentration profile of the Raman-active substance after and before the application of the skin care composition; and the thickness of the stratum corneum after and before the application of the skin care composition.

WO18029673 A1 (Ahava Dead Sea Laboratories Ltd) discloses compositions comprising at least one Dead Sea extract and at least one polysaccharide, wherein the repeating unit of said polysaccharide is a tetrasaccharide consisting of two D-glucose residues, one L-fucose residue and one D-glucuronic acid residue. The formulations are useful as skin protectants against pollution.

US20020071820 A1(L'Oreal) discloses the use of cubic gel particles as an antipollution agent for protecting the body against the effects of pollution, which includes applying to the keratin material a composition containing an effective amount of cubic gel particles in a physiologically acceptable medium. The cubic gel particles are preferably in aqueous dispersion and are preferably formed either from a compound chosen from 3,7,11,15-tetramethyl-1,2,3-hexadecanetriol, phytanetriol, N-2-alkoxycarbonyl derivatives of N-methylglucamine and unsaturated fatty acid monoglycerides, and from a dispersing and stabilizing agent, or from a mixture of at least two amphiphilic compounds, one of the amphiphilic compounds preferably being capable of forming a lamellar phase in the presence of water and the other preferably being capable of forming an inverse hexagonal phase in the presence of water.

Despite the methods disclosed hereinabove, there is need for a robust in vitro method to determine, as accurately as possible, the short-term and the long-term efficacy of cosmetic compositions which prevent, or at least delay, the contact of particulate atmospheric pollutants with human skin by forming a protective layer between the surface of the skin and the pollutant. Such information could be useful to formulate more efficacious compositions, or to be able to choose one composition over few others in view of their efficacy.

The present invention addresses the needs by overcoming at least one drawback, disadvantage or limitation of the state of the art.

### SUMMARY OF THE INVENTION

In accordance with a first aspect is disclosed an in vitro method to determine efficacy of a cosmetic composition or of one or more ingredients comprised therein to inhibit a particulate pollutant from contacting skin, said method comprising the steps of:
(i) contacting a human skin equivalent with a cosmetic composition to form, upon drying, a layer thereof extending along mutually perpendicular X and Y and Z axis where along said Z axis said layer is 1 to 100 μm, where said Z axis indicates thickness of said layer;
(ii) depositing, on said layer, a known amount of a model fine particulate matter comprising a first substance responsive to depth profile analysis where said cosmetic composition comprises a second substance, not comprised in said model fine particulate matter but also responsive to said depth profile analysis where the responses of said first and second substances are distinguishable;
(iii) for a pre-determined period after depositing said model fine particulate matter, periodically measuring response of said first and said second substance by said depth profile analysis, to thereby determine the amount of said model fine particulate matter at defined intervals along said Z axis; and,
(iv) ascertaining said efficacy based on the amount of said model fine particulate matter at said defined intervals along said Z axis throughout said pre-determined period.

In accordance with a second aspect is disclosed a process of demonstrating efficacy of a cosmetic composition or an active ingredient comprised therein to inhibit an atmospheric pollutant from contacting skin, comprising the steps of:
(i) contacting a human skin equivalent with a cosmetic composition to form, upon drying, a layer thereof extending along mutually perpendicular X and Y and Z axis where along said Z axis said layer is 1 to 100 μm, where said Z axis indicates thickness of said layer;
(ii) depositing, on said layer, a known amount of a model fine particulate matter comprising a first substance responsive to depth profile analysis where said cosmetic composition comprises a second substance, not comprised in said model fine particulate matter but also responsive to said depth profile analysis where the responses of said first and second substances are distinguishable;
(iii) for a pre-determined period after depositing said model fine particulate matter, periodically measuring response of said first and said second substance by said depth profile analysis, to thereby determine the amount of said model fine particulate matter at defined intervals along said Z axis; and,
(iv) ascertaining and thereby demonstrating said efficacy based on the amount of said model fine particulate matter at said defined intervals along said Z axis throughout said pre-determined period.

### DETAILED DESCRIPTION OF THE INVENTION

As used herein the term “comprising” encompasses the terms “consisting essentially of” and “consisting of”. Where the term “comprising” is used, the listed steps or options need not be exhaustive. Unless otherwise specified, numerical ranges expressed in the format “from x to y” are understood to include x and y. In specifying any range of values or amounts, any particular upper value or amount can be associated with any particular lower value or amount. Except in the examples and comparative experiments, or where otherwise explicitly indicated, all numbers are to be understood as modified by the word “about”. All percentages and ratios contained herein are calculated by weight unless otherwise indicated. As used herein, the indefinite article “a” or “an” and its corresponding definite article “the” means at least one, or one or more, unless specified otherwise. The various features of the present invention referred to in individual sections above apply, as appropriate, to other sections mutatis mutandis. Consequently, features specified in one section may be combined with features specified in other sections as appropriate. Any section headings are added for convenience only, and are not intended to limit the disclosure in any way. The invention is not limited to the embodiments illustrated in the drawings. The examples are intended to illustrate the invention and are not intended to limit the invention to those examples per se.

The term cosmetic composition means any cosmetic composition. More particularly the cosmetic composition is a leave-on cosmetic. “Leave-on” as used herein means compositions that are applied to the skin and are not intended to be washed or rinsed off for some time, as contrasted with cleansing or wash-off or rinse-off compositions.

Preferably the leave-on cosmetic composition is a serum, hand creams, face creams, body lotions, make up compositions such as foundations, lipsticks, hair styling gels, hair styling creams and deodorants and antiperspirants such as roll on or sticks. The compositions may accordingly be in a variety of formats as described hereinbefore.

The term in vitro means that the method in accordance with the invention is not carried out on human volunteers, for example, on forearms of human volunteers.

The term active ingredient means any ingredient, including film forming polymers included in the cosmetic composition to inhibit or prevent the contact of particulate pollutants with human skin. Non-limiting examples thereof include silicone polymers and extracts of natural products such as extract of roots or leaves of any plant.

Human skin acts like a natural shield which protects our body from external influences. However, at times, and under certain conditions, the skin may no longer perform this function fully and efficiently. There is plethora of evidence to substantiate that atmospheric pollutants affect the normal functioning of human skin. Particulate pollutants tend to top the list at least in some countries or some regions of the world. Formulation scientists have explored and continue to explore newer and more effective cosmetic compositions to protect the skin from particulate pollutants, including the compositions or active agents which can resist, restrict or prevent the contact of such pollutants with skin. However, as discussed at length under the section of background and prior art, there is need for a more robust and reliable method for demonstrating the efficacy of such compositions. The present invention addresses such as need, at least in part.

The term particulate pollutant, also called particulate matter or PM, means a mixture of solids and liquid droplets floating in the air. Some particles are released directly from a specific source, while others form in complicated chemical reactions in the atmosphere. Suitable examples include dust, dirt, soot, or smoke. Particulate pollutants are described in terms of particle size: PM_{2.5} and PM₁₀ having an aerodynamic diameter less than 2.5 μm and 10 μm, respectively. It is preferred that in the method in accordance with the invention, the model fine particulate matter resembles PM_{2.5} or PM₁₀ at least in size.

The method in accordance with the invention is carried out on a human skin equivalent, i.e. a material that resembles the skin of human beings. Preferably the human skin-equivalent is artificial skin, or a living skin equivalent. Further preferably it is a regenerated tissue. Currently, several HSEs are commercially available for such applications. Examples include Apligraf®, Epicel®, Dermagraft®, Alloderm®, Transcyte®, Orcel®, Integra® DRT, Epistem® and StrataGraft®. These HSEs can be divided into three major categories: epidermal, dermal, and full-thickness models. A particularly preferred human skin-equivalent is Vitro-Skin®. It is a type of polymeric artificial skin. Other equivalents include cellulose nitrate, nylon or PTFE membrane.

The first step involves contacting the human skin equivalent with a cosmetic composition to form, upon drying, a layer thereof extending along mutually perpendicular X and Y and Z axis where along said Z axis said layer is 1 to 100 μm, where said Z axis indicates thickness of said layer. Along the X and Y axis it is acceptable so long as the area occupied by the layer is adequate to permit the analysis.

It may become necessary to be able to determine as accurately as possible, the dynamics of penetration of a model pollutant from the atmosphere over a period so that is thereby possible to determine the short-term efficacy and the long-term efficacy of cosmetic compositions that claim to prevent, or at least delay the contact of particulate atmospheric pollutants with human skin by forming to the extent possible, a barrier between the skin and the pollutant. Usually, after elapse of time upon application of a leave-on cosmetic composition to the skin, the composition forms a layer on the skin, which performs the intended function. When such a cosmetic is intended to act as a barrier against environmental pollutants such as PM_{2.5}, in the initial stage the layer effectively prevents or resists the contact of the pollutant with the skin. However, over a period, the pollutant may enter the bulk of the layer and then try to migrate towards the skin. The layer will resist this migration, to the extent it can, depending on the nature of the composition and the efficacy of active ingredient therein, if any, such as film forming polymers. Eventually the pollutant may even pass through the layer and establish contact with the skin.

Preferably the amount is equivalent to the amount applied under in use conditions. Usually cosmetic compositions comprise water, other volatile solvents or oils. Water and other volatile solvents evaporate upon application of the composition to the skin. On the other hand, where the compositions comprise oils, the oils tend to be absorbed by the skin. Eventually the composition dries up and leaves behind a film or layer on the skin, which, depending on the amount applied or the recommended amount for application, could range from a few microns to few thousand microns.

The next step involves simulating the contact of human skin with a model particulate pollutant. This is done by depositing, on said layer, a known amount of a model fine particulate matter comprising a first substance responsive to depth profile analysis where said cosmetic composition comprises a second substance not comprised in said model fine particulate matter but also responsive to said depth profile analysis where the response of said first and second substances are distinguishable. The response of the first and the second substance will vary depending on the nature of the substance, for example, in the case of Confocal Microscopy, the response will be measured as the intensity of fluorescence and the distinction could be made based on the wavelength of the emitted radiation.

The next step involves, for a pre-determined period after depositing said model fine particulate matter, periodically measuring response of said first and said second substance by said depth profile analysis, to thereby determine the amount of said model fine particulate matter at defined intervals along said Z axis. The pre-determined period is preferably from 1 to 480 minutes, more preferably 1 to 240 minutes and it is governed by the time taken for the experiments and the nature of the analytical method.

It is preferred that the depth profile analysis is done by Confocal Raman Spectroscopy, Confocal Laser Scanning Microscopy, Time of Flight Secondary Ion Mass Spectrometry, Optical Coherence Tomography, XPS, Glow Discharge Mass Spectrometry, Glow Discharge Optical Emission Spectroscopy, Laser-Ablated Ion Coupled Plasma—Mass Spectroscopy or Secondary Ion Mass Spectrometry and said substance is correspondingly responsive to said depth profile analysis. More preferably said depth profile analysis is done by Confocal Laser Scanning Microscopy or Confocal Raman Spectroscopy and said substance is correspondingly responsive to said depth profile analysis.

It is preferred that the model fine particulate matter comprises a synthetic polymeric material, a natural polymeric material, a water-insoluble salt, a mineral, a metal, an alloy, glass or a mixture thereof. Further preferably the synthetic polymeric material is a polyamide, polyacetate, polyester, polyacrylate, polystyrene, polyethylene, polypropylene, rayon, polyvinyl chloride or a mixture thereof. Further preferably the natural polymeric material is cellulose, regenerated cellulose, starch, microcrystalline cellulose or a mixture thereof. It is particularly preferred that the model fine particulate matter is in the form of beads comprising polystyrene and a fluorescent material, the microscopic imaging technique is fluorescence microscopy. In such a case it is preferred that diameter of the model fine particulate matter is 10 nm to 50000 nm. This enables easier experimentation. Further preferably the fluorescent material absorbs and emits radiation of wavelength 500 to 2500 nm. In such a case it is preferred that the intensity of fluorescence of emitted radiation is measured in-line with the excitation radiation.

A particularly preferred material is Fluorescent Probes, which are Polystyrene-based particles (1 μm diameter) with fluorescent tagging Ex. Polysciences Inc. It is preferred that the fluorescent material absorbs and emits radiation of wavelength 400 to 700 nm. Further preferably the signals of fluorescence are recorded in two channels, a first channel in the range of 470 to 550 nm (green) and a second channel in the range of 590 to 700 nm (red). In a further preferred aspect, 0.2 μg/cm² to 20 μg/cm² of the model-pollutant is deposited on said layer. To simulate actual real-life situation to the extent possible, the amount of the model particulate matter should be enough to simulate such conditions.

Preferably images along said Z axis, indicative of said amount of said model particulate matter are collected at intervals of 0.1 to 1 μm to generate plurality of stacks of images. It is further preferred that thickness of each said stack is 5 to 8 μm.

Once all the required images have been acquired, it is preferred that the images are analysed by an image analysis software to determine the number of particles of said model pollutant at each successive interval, where the number of said particles at each successive interval is indicative of its amount at each said interval. Preferably the software is ImageJ. Alternatively, any equivalent software could be used.

As far as the efficacy of the candidate cosmetic composition is concerned, it is preferred that the efficacy is measured as the percentage amount of the model fine particulate matter that has penetrated the layer at the end of the pre-determined period as compared to the total amount thereof at the beginning of the period. Preferably, a composition is deemed efficacious if the percentage is less 30%. Further preferably a composition is deemed highly efficacious if, at the end of said pre-determined period, less than 10% of said model fine particulate matter has reached an interval within 2 μm reckoned from the end of said layer which is proximate to the artificial skin.

The method of the invention is used to determine either the efficacy of a candidate cosmetic composition as a whole, or of one or more active ingredients in the composition. Preferably the said one or more ingredients is a film-forming polymer. An example thereof is MQ® Resins.

The method in accordance with this invention can be used to distinguish between efficacy of a first composition from a second different composition. In this case, the steps (i) to (iii) are first carried out on a first composition and then repeated on a second different composition to thereby compare the efficacy of said first composition with said second.

Alternatively, the first composition comprises an amount of a film-forming polymer and said second composition comprises a different amount of the same film-forming polymer, where said first and said second compositions are identical except for said amount. Preferably the first composition comprises an amount of a first ingredient and said second composition comprises either the same amount or different amount of another ingredient.

The demonstration in accordance with the invention may be useful for any consumer promotion event, or a consumer demonstration such as in a mall or supermarket or a consumer fair. The demonstration may also be useful for claim support and advertising.

The method provides reliable and results at much lower pollutant dosage of approximately 0.25 μg/cm² of skin which better represents a real-life situation of skin exposed to pollutants, especially particulate pollutants.

### The Composition

The compositions can be applied directly to the skin. Alternatively, they can be delivered by various transdermal delivery systems, such as transdermal patches as known in the art. For example, for topical administration, the active ingredient can be formulated in a solution, gel, lotion, ointment, cream, suspension, paste, liniment, powder, tincture, aerosol, patch, or the like in a cosmetically acceptable form by methods known in the art. The composition can be any of a variety of forms common in the cosmetic arts for topical application to humans.

The compositions may be made into a wide variety of product types that include but are not limited to solutions, suspensions, lotions, creams, gels, toners, sticks, sprays, ointments, pastes, foams, powders, mousses, strips, patches, electrically-powered patches, hydrogels, film-forming products, facial and skin masks, make-up such as foundations and the like. These product types may contain several types of cosmetically-acceptable carriers including, but not limited to solutions, suspensions, emulsions such as microemulsions and nanoemulsions, gels, solids and liposomes.

The compositions can be formulated as solutions which include an aqueous or organic solvent, e.g., 50 to 90 wt % of a cosmetically acceptable aqueous or organic solvent. Examples of suitable organic solvents include: propylene glycol, polyethylene glycol (200-600), polypropylene glycol (425-2025), glycerol, 1,2,4-butanetriol and sorbitol esters.

A lotion can be made from such a solution. Lotions typically contain from about 1% to about 20 wt % emollient(s) and from 50 to 90 wt % water.

Another type of product that may be formulated from a solution is a cream. A cream typically contains 5 to 50 wt % emollient(s) and 45 to 85 wt % water.

The compositions described herein can also be formulated as emulsions. If the carrier is an emulsion, then 1 to 10 wt % of the carrier contains an emulsifier(s). Emulsifiers may be nonionic, anionic or cationic.

Lotions and creams can be formulated as emulsions. Typically, such lotions contain 0.5 to 5 wt % emulsifier(s), while such creams typically contain 1% to 20 wt % emulsifier(s).

Single emulsion skin care preparations, such as lotions and creams, of the oil-in-water type and water-in-oil type are well-known in the art and are useful in compositions and methods described herein. Multiphase emulsion compositions, such as the water-in-oil-in-water type or the oil-in-water-in-oil type, are also useful in the compositions and methods describe herein. In general, such single or multiphase emulsions contain water, emollients, and emulsifiers as essential ingredients.

The compositions described herein can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contain 0.1 to 5 wt % gelling agents.

Cosmetic compositions described herein may typically comprise a derivative of any compound or composition described herein and optionally, a polar solvent. Solvents suitable for use in the formulations described herein include any polar solvent capable of dissolving the derivative. Suitable polar solvents may include: water; alcohols (such as ethanol, propyl alcohol, isopropyl alcohol, hexanol, and benzyl alcohol); polyols (such as propylene glycol, polypropylene glycol, butylene glycol, hexylene glycol, maltitol, sorbitol, and glycerine); and panthenol dissolved in glycerine, flavor oils and mixtures thereof. Mixtures of these solvents can also be used. Exemplary polar solvents may be polyhydric alcohols and water. Examples of solvents may include glycerine, panthenol in glycerine, glycols such as propylene glycol and butylene glycol, polyethylene glycols, water and mixtures thereof. Additional polar solvents for use may be alcohols, glycerine, panthenol, propylene glycol, butylene glycol, hexylene glycol and mixtures thereof.

An emollient may also be added. The emollient component can comprise fats, oils, fatty alcohols, fatty acids and esters which aid application and adhesion, yield gloss and provide occlusive moisturization. Suitable emollients for use may be isostearic acid derivatives, isopropyl palmitate, lanolin oil, diisopropyl dimerate, maleated soybean oil, octyl palmitate, isopropyl isostearate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl linoleate, pentaerythrityl tetrastearate, neopentylglycol dicaprylate/dicaprate, hydrogenated coco-glycerides, isononyl isononanoate, isotridecyl isononanoate, myristyl myristate, triisocetyl citrate, cetyl alcohol, octyl dodecanol, oleyl alcohol, panthenol, lanolin alcohol, linoleic acid, linolenic acid, sucrose esters of fatty acids, octyl hydroxystearate and mixtures thereof. Examples of other suitable emollients can be found in the Cosmetic Bench Reference, (1996), or in the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7.sup.th Edition, 1997) (hereinafter “ICI Handbook”).

Other suitable solid/liquid agents may include vitamins and their derivatives. Compositions of the present invention may include vitamins as the desired active. Illustrative vitamins are Vitamin A (retinol) as well as retinol esters like retinol palmitate and retinol propionate, Vitamin B2, Vitamin B3 (niacinamide), Vitamin B6, Vitamin C, Vitamin D, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocotrienol, tocopheryl palmitate and tocopheryl linoleate. DL-panthenol and derivatives may also be employed. Total amount of vitamins when present in the compositions may range from 0.001 to 10%.

Sunscreen agents may also be included in compositions of the present invention as solid/liquid agents. Particularly preferred are such materials as phenylbenzimidazole sulfonic acid (Ensulizole), ethylhexyl salicylate (octyl salicylate), ethylhexyl p-methoxycinnamate, available as Parsol MCX®, Avobenzene, available as Parsol 1789® and benzophenone-3, also known as Oxybenzone®. In addition, Octocrylene® is also suitable. Amounts of the sunscreen agents when present may generally range from 0.1 to 30 wt %.

Suitable oils include esters, triglycerides, hydrocarbons and silicones. These can be a single material or a mixture of one or more materials. They may normally comprise 0.5 to 90 wt %.

Examples of surface active agents which may be used in the compositions described herein include sodium alkyl sulfates, e.g., sodium lauryl sulfate and sodium myristyl sulfate, sodium N-acyl sarcosinates, e.g., sodium N-lauroyl sarcosinate and sodium N-myristoyl sarcosinate, sodium dodecylbenzenesulfonate, sodium hydrogenated coconut fatty acid monoglyceride sulfate, sodium lauryl sulfoacetate and N-acyl glutamates, e.g., N-palmitoyl glutamate, N-methylacyltaurin sodium salt, N-methylacylalanine sodium salt, sodium alpha-olefin sulfonate and sodium dioctylsulfosuccinate; N-alkylaminoglycerols, e.g., N-lauryl-diamino-ethylglycerol and N-myristyldiaminoethylglycerol, N-alkyl-N-carboxymethylammonium betaine and sodium 2-alkyl-1-hydroxyethylimidazoline betaine; polyoxyethylenealkyl ether, polyoxyethylenealkylaryl ether, polyoxyethylenelanolin alcohol, polyoxyethyleneglyceryl monoaliphatic acid ester, polyoxyethylenesorbitol aliphatic acid ester, polyoxyethylene aliphatic acid ester, higher aliphatic acid glycerol ester, sorbitan aliphatic acid ester, Pluronic type surface active agent, and polyoxyethylenesorbitan aliphatic acid esters such as polyoxyethylenesorbitan monooleate and polyoxyethylenesorbitan monolaurate. Emulsifier-type surfactants known to those of skill in the art can be used in the compositions described herein.

The surfactants can be used at levels from 4 to 90% depending on the type of the composition.

In addition, these compositions may include therapeutic agents, carriers, adjuvants, and the like. Some particular additional agents may include retinoids; antioxidants; hydroxy acids; fatty acids, acceptable non-toxic metal salts of naturally occurring amino acids or of hydroxyalkyl acids; botanical extracts, salicylic acid, keratolytic agents, complexing agents, colorants and fragrance ingredients.

The invention will now be described in detail with the following non-limiting examples.

### EXAMPLES

### Example 1

Fluoresbrite® YG carboxylate microspheres (2.5% aqueous suspension), from Polysciences, were used as model PM 2.5, which are monodispersed polystyrene particles with nominal diameters of 0.50 μm. Their maximum excitation and emission spectrums are 441 nm and 486 nm respectively. Nile red (Molecular Probe, UK), known to fluoresce strongly in hydrophobic environment was used to stain oil phase of skin cream to indicate where cream film stands after water evaporates. Its excitation and emission spectrums are 559 nm and 637 nm respectively.

Glass slides of a smooth surface was used as substrate for film application for initial test, as well as VITRO-SKIN® (from IMS—USA Portland Me., IMS Inc.) of certain roughness, which mimics the surface properties of human skin.

Silicone resin MQ1640 (MQ) from Dow corning was chosen to test its block efficacy, as it is a well-recognized polymer for its film forming properties.

The following two cosmetic creams (o/w emulsion based) were prepared.

TABLE 1

Composition Ref/wt %

| Ingredient | Control | A |
|---|---|---|
| DC 245 | 8.0 | 8.0 |
| Capric Caprylic Triglycerides | 3.0 | 3.0 |
| Glyceryl Stearate | 1.0 | 1.0 |
| Aristoflex ® AVC | 1.0 | 1.0 |
| Mryj ® 59P | 1.8 | 1.8 |
| MQ1640 | — | 3.0 \| |
| Water and other minors to 100 wt % | Balance | Balance |

Product films of vehicle (check vehicle base) base, with or without MQ were applied on VITRO-SKIN® by a cubic film applicator with thickness of 75 μm (film thickness after drying=7 microns). The film was left dried for at least 30 minutes. Fluoresbrite® YG carboxylate microspheres suspension was diluted 80 times by DI water [13]. The diluted suspension was filled in a plastic cosmetic spray bottle. Continuously press the spray button until a steady flow of liquids form. A constant distance of 5 cm was kept of the spray bottle from the target product film described in section 2.3. And then, two sprays were applied on the target. Samples were incubated for 3 hours. The dosage of model particulate pollutant was 1 μg/cm².

Images were obtained using a Leica SP5 laser scanning confocal microscope. Samples were excited using 488 nm (Argon) laser (20% intensity). A 50×/dry objective was selected to observe the samples. Fluorescent signals were recorded in two discrete channels at 470550 nm (green) and 590-700 nm (red) respectively. The zoom factor was set to 4 to detect 500 nm particles more clearly. Regions of interest were randomly selected in a 73.8×73.8 μm² field of view. Images in the z-direction were collected every 0.5 μm to generate an image stack. Each image stack recorded was 5 to 8 μm deep. At least three random regions of interest were recorded for each cream film sample to prevent selection bias. Furthermore, the entire image acquired was subjected to the analysis described below.

There are difficulties in using VITRO-SKIN®, because it is not flat. Some of fields of view selected showed a sloping cream surface across the image, which will bring challenges in image analysis. To mitigate such difficulties, VITRO-SKIN® was fixed by double sided adhesive stripes on a microscope glass slide after cream film was applied and dried. And areas of interests were carefully selected to avoid a sloping sample. Similarly, deeper stacks of images were used for particle number analysis to avoid impacts from surface roughness. Particle number versus skin cream depth on VITRO-SKIN® in a field of viewing of 73.8×73.8 μm². A zoom-in area of the selected area on the left side. The point where skin cream coverages reach a relatively stable value were set as skin depth zero. And then every 0.5 μm into the sample in the Z-axis was recorded until red fluorescence (cream film) was no longer visible. The error bars generated were from n=3. Total number of particles that were applied topically onto the skin cream film were the sums of particle numbers calculated in each layer.

The observations are summarised in Table 2.

TABLE 2

| | |
|---|---|
| Interval/μm reckoned from the | Number of particles of |
| end of said layer which is | the model pollutant |

| proximate the VITRO-SKIN ® | Control | A |
|---|---|---|
| 0 | 420 | 850 |
| 0.5 | 100 | 40 |
| 1.0 | 100 | 10 |
| 1.5 | 80 | 10 |
| 2.0 | 100 | ~0 |

The total numbers of particles applied topically on cream film with and without MQ are similar to each other. And in the case of no MQ addition, about 12% (100 out of 840) can reach the substrate, while in the case of 3% MQ addition, no particles were found to reach the substrate. The difference is significant.

The illustrated examples clearly indicate that the method in accordance with the invention is a robust and reliable tool for measuring and comparing the efficacy of cosmetic cleansing compositions to remove particulate pollutants adhering to the skin. The method could be used to measure and demonstrate cleansing efficacy of cosmetic cleansing compositions against atmospheric pollutants, especially particulate pollutants such as PM_{2.5} and PM₁₀. The testing could be monadic, alternatively paired-testing or further alternatively discrete-choice testing. Outcome of the method could potentially be useful for relative ranking of various cleansing compositions belonging to the same category of products, such as a soap-based cleanser v/s a non-soap surfactant-based cleanser, or even, where necessary, between two or more products belonging to different categories of products for example, a shampoo against a soap bar. The method could also be useful to determine the efficacy of one or more active ingredients such as surfactants and polymers by suitably formulating the candidate compositions to be tested. Further, the method of the invention could also be used as a demonstration tool for consumer promotion or activation of new or existing cosmetic cleansing compositions. Outcome of the method could also be used for claim-support.

1. An in-vitro method to determine efficacy of a cosmetic composition or of one or more ingredients comprised therein to inhibit a particulate pollutant from contacting skin, said method comprising the steps of:
contacting a human skin equivalent with a cosmetic composition to form, upon drying, a layer thereof extending along mutually perpendicular X and Y and Z axis where along said Z axis said layer is 1 to 100 μm, where said Z axis indicates thickness of said layer;
(ii) depositing, on said layer, a known amount of a model fine particulate matter comprising a first substance responsive to depth profile analysis where said cosmetic composition comprises a second substance not comprised in said model fine particulate matter but also responsive to said depth profile analysis where the responses of said first and second substances are distinguishable;
(iii) for a pre-determined period after depositing said model fine particulate matter, periodically measuring response of said first and said second substance by said depth profile analysis, to thereby determine the amount of said model fine particulate matter at defined intervals along said Z axis; and,
(iv) ascertaining said efficacy based on the amount of said model fine particulate matter at said defined intervals along said Z axis throughout said pre-determined period.

2. The method as claimed in claim 1, wherein said model fine particulate matter resembles PM_{2.5} or PM₁₀ at least in size.

3. The method as claimed in claim 1, wherein said depth profile analysis is done by Confocal Raman Spectroscopy, Confocal Laser Scanning Microscopy, Time of Flight Secondary Ion Mass Spectrometry, Optical Coherence Tomography, XPS, Glow Discharge Mass Spectrometry, Glow Discharge Optical Emission Spectroscopy, Laser-Ablated Ion Coupled Plasma—Mass Spectroscopy or Secondary Ion Mass Spectrometry and said first and second substance is correspondingly responsive to said depth profile analysis.

4. The method as claimed in claim 3, wherein said depth profile analysis is done by Confocal Laser Scanning Microscopy or Confocal Raman Spectroscopy.

5. The method as claimed in claim 1, wherein said human skin equivalent is artificial skin, donor skin, living skin equivalent or pig skin.

6. The method as claimed in claim 5, wherein said artificial skin is a polymer-based substrate mimicking human skin.

7. The method as claimed in claim 1, wherein said model fine particulate matter comprises a synthetic polymeric material, a natural polymeric material, a water-insoluble salt, a mineral, a metal, an alloy, glass or a mixture thereof.

8. The method as claimed in claim 7, wherein said synthetic polymeric material is a polyamide, polyacetate, polyester, polyacrylate, polystyrene, polyethylene, polypropylene, rayon, polyvinyl chloride or a mixture thereof.

9. The method as claimed in claim 1, wherein images along said Z axis, indicative of said amount of said model particulate matter are collected at intervals of 0.1 to 10 μm to generate plurality of stacks of images.

10. The method as claimed in claim 9, wherein thickness of each said stack is 5 to 8 μm.

11. The method as claimed in claim 9, wherein said images are analyzed by an image analysis software to determine the number of particles of said model pollutant at each successive interval, where the number of said particles at each successive interval is indicative of its amount at each said interval.

12. The method as claimed in claim 1, wherein said efficacy is measured as the percentage of said model fine particulate matter inside said layer at the end of said pre-determined period as compared to the total amount thereof at the beginning of said period.

13. The method as claimed in claim 12, wherein a composition is deemed efficacious if said percentage is less 30%.

14. The method as claimed in claim 1, wherein said model fine particulate matter is in the form of beads comprising polystyrene and a fluorescent material.

15. A process of demonstrating efficacy of a cosmetic composition or an active ingredient comprised therein to inhibit an atmospheric pollutant from contacting skin, comprising the steps of:
contacting a human skin equivalent with a cosmetic composition to form, upon drying, a layer thereof extending along mutually perpendicular X and Y and Z axis where along said Z axis said layer is 1 to 100 μm, where said Z axis indicates thickness of said layer;
(ii) depositing, on said layer, a known amount of a model fine particulate matter comprising a first substance responsive to depth profile analysis where said cosmetic composition comprises a second substance, not comprised in said model fine particulate matter but also responsive to said depth profile analysis where the responses of said first and second substances are distinguishable;
(iii) for a pre-determined period after depositing said model fine particulate matter, periodically measuring response of said first and said second substance by said depth profile analysis, to thereby determine the amount of said model fine particulate matter at defined intervals along said Z axis; and,
(iv) ascertaining and thereby demonstrating said efficacy based on the amount of said model fine particulate matter at said defined intervals along said Z axis throughout said pre-determined period.

16. The method as claimed in claim 1, wherein the pre-determined period ranges from 1 to 480 minutes.

17. The method as claimed in claim 7, wherein said natural polymeric material is cellulose, regenerated cellulose, starch, microcrystalline cellulose or a mixture thereof.
